(19) United States
(12) Patent Application Publication
(10) Pub. No.: US 2007/0244384 A1
(43) Pub. Date: Oct. 18, 2007

(54) Patient calming arrangements

(75) Inventor: Russell Peter Gore, Abingdon (GB)

Correspondence Address: CROWELL & MORING LLP;INTELLECTUAL PROPERTY GROUP, P.O. BOX 14300, WASHINGTON, DC, 20044-4300, US

(73) Assignee: Siemens Magnet Technology Ltd., Oxon (GB)

(21) Appl. No.: 11/723,534

(22) Filed: Mar. 20, 2007

(30) Foreign Application Priority Data
Mar. 21, 2006 (GB) 0605641.0

(51) Int. Cl. A61B 6/00

(52) U.S. Cl. 600/407

(57) ABSTRACT

An arrangement for presenting visual images, for example to a patient to be investigated by a scanning medical apparatus of the kind having a patient volume intended to accommodate part at least of the patient; the arrangement comprising visual display means including at least one screen so disposed in relation to said apparatus as to display said images during approach of the patient to and/or during scanning of the patient by said apparatus; and means for supplying said at least one screen with images derived from part at least of the environment of said scanning apparatus, such that the images displayed to the patient reveal features of said environment which the patient would see directly, were the scanning apparatus not there.

This invention relates to arrangements for calming patients during investigation by certain medical systems, such as MRI or CT scanning, which may involve the confinement of a patient within, and/or movement of a patient through, a patient volume which may appear tunnel-like to some patients, and thus give rise to apprehension or even trigger panic attacks. The patient volume may be a tubular bore in scanners using a solenoidal magnet, or may be a more open volume in scanners having other architectures, such as C- H- or ‘Temple-’ shaped magnets, known to those skilled in the art.

The psychological effect upon patients during their approach to, and during investigation by such exploratory systems is a primary driver in MRI system design. In particular, claustrophobic reactions can significantly reduce patient throughput, either through refusal to enter the system or by ‘pressing the panic button’ during the scanning process. This results in extended scan times and can further discomfort patients who may be already distressed as a result of the condition for which they are being examined. As a secondary result, the reduction in patient throughput can increase operating costs for the system providers.

Typical strategies to minimise this discomfort and disruption involve making the patient volume of the imaging system as axially short and as open as possible. Such strategies, however, tend to have significant and adverse impacts on system cost and performance. The present invention attempts to provide an environment that reduces the feeling of confinement suffered by the patient during scanning and/or on approaching the system, hence reducing anxiety, trauma and delays.

United Kingdom patent application GB2286887 describes a medical scanning apparatus wherein the housing surrounding the aperture is shaded or otherwise marked so as to produce a visual perspective effect which makes the aperture appear larger than it actually is, so as to reduce feelings of claustrophobia in patients.

The present invention accordingly provides apparatus as defined in the appended claims.

According to one aspect of the invention, there is provided an arrangement for presenting visual images to a patient to be investigated by medical apparatus of the kind having a patient volume intended to accommodate part at least of the patient; the arrangement comprising visual display means including at least one screen so disposed in relation to said apparatus as to display said images during approach of the patient to and/or to the patient when at least partially accommodated within the patient volume. Preferably, said images comprise, in part at least, visual information relating to the environment of said apparatus and in particular convey to the patient images of parts of said environment which are otherwise obscured by the scanning apparatus.

The said at least one screen may conveniently comprise one or more display screens so installed at the entrance to, and/or within, a patient volume of the apparatus through or into which the patient is installed during examination. Preferably, the or each display screen is a thin, flat screen, such as an LCD or plasma screen.

In preferred embodiments of the invention, the images shown on the screen or screens may be used to promote the appearance of system openness by displaying images of the immediate environment of the medical apparatus derived from appropriately positioned cameras to convey, for example, the impression that part at least of the apparatus, in the vicinity of the patient volume, is not there, and/or by showing psychologically calming images, such as colour fields, concentric circles or radial patterns, or sylvan scenes.

Alternatively, some patients may be calmed more readily by displaying images which engage their attention, such as an artistic or sporting performance or, for children, a cartoon, and which distracts such patients from their immediate surroundings.

One embodiment of the invention, for example, deploys an annular screen, or several sector-shaped component screens, arranged to surround the entrance to the patient volume. The screen or screens may be fed directly with images from one or more cameras looking away from the system, or with previously recorded images obtained from such a camera or cameras, such that the region covered by the display appears substantially transparent or invisible, giving the patient the impression of a widened entrance to said patient volume. Such an arrangement can reduce patient apprehension on approaching the system.

It is preferred in such circumstances that the room environment beyond the scanning apparatus, as approached by the patient, bears one or more strong visual cues, such as a strategically placed window or picture and/or bright colouring or a strong pattern which, when partially reproduced as an image on the screen or screens, will be focussed upon by the patient and can help establish an illusion of transparency or invisibility in at least a region of the apparatus surrounding an opening to the patient volume.

In some embodiments, it is preferred that the video signals from one or more cameras or recorded image reproducing devices are subjected to geometric processing to allow for differences in parallax, scale and/or orientation, perceived by the patient, as between the real environment and the image(s) shown on the screen or screens. Correction may also be provided to account for the differences in focal lengths between the cameras and the eyes of the patient.

It is further preferred, in some embodiments of the invention, to subject said video signals to temporal processing, to accommodate changes in lighting or similar visual events in the examination room.

Alternatively to the use of captured images, artificial images such as computer generated images may be produced and supplied to the screen(s) so as to be perceived by the patient.

Alternatively, or in addition to one or more screens mounted around its entrance, the patient volume can accommodate one or more screens, extending along the axis of the patient volume, and which can be fed with images captured from one or more external cameras, or computer-generated images or images from a recording of any convenient kind, such that the image displayed to the patient shows that which the patient would see were the imaging system not there. The feeling of apparent openness diminishes claustrophobic reaction and thus enhances scanning efficiency.

Again, the video signals supplied to the screen or screens from whatever source may be subjected to geometric and/or temporal processing as described above.

Such display devices mounted in the patient volume can also be useful in functional MRI studies where the response of a patient to the display of appropriate visual stimuli is material to the complaint under investigation.

When not in use for calming patients or generating visual stimuli, the screen or screens may be utilised to display other material, such as advertisements or company logos for example, enabling ‘sponsorship funding’ of the purchase or operation of the equipment to acknowledged.

In order that the invention may be clearly understood and readily carried into effect, certain embodiments thereof will now be described, by way of example only, with reference to the accompanying drawings, of which:

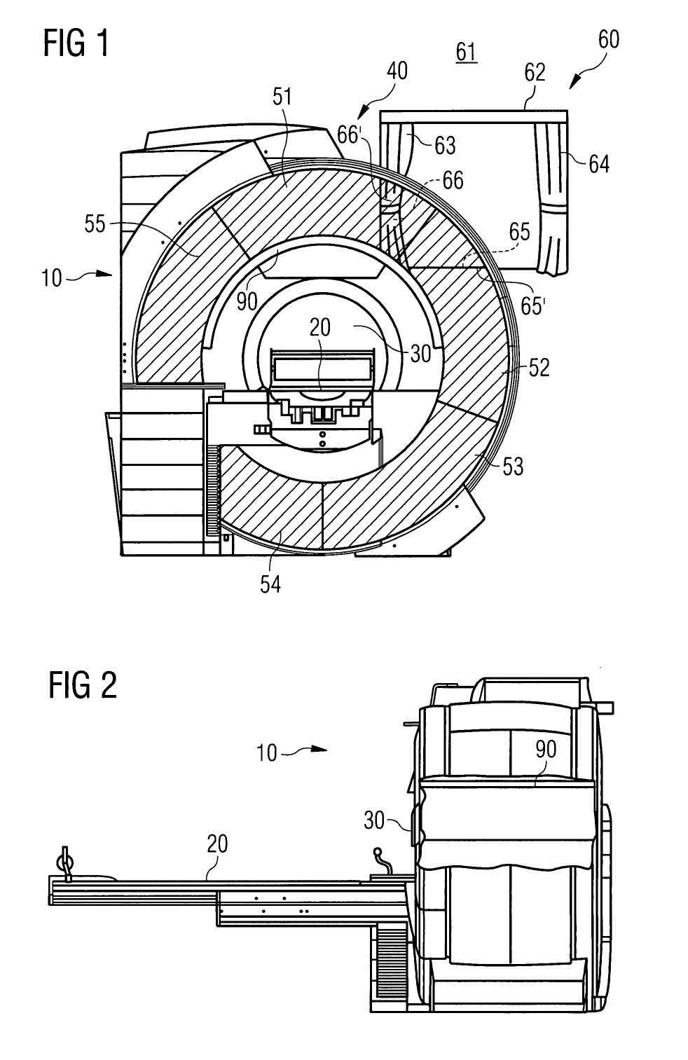

FIG. 1 shows, in frontal elevation, a scanner arrangement in accordance with one example of the invention; and

FIG. 2 shows a schematic and partial cross-section through the scanner arrangement of FIG. 1.

Referring now to FIG. 1, there is shown an MRI scanner 10 including a patient support 20 disposed in a patient volume 30 of the scanner, through which the patient will be moved for examination, whilst lying on the support 20. It will be appreciated that the patient is moved axially of the patient volume 30, and that the patient support 20 extends in that axial direction.

While the present invention may usefully be applied to any medical system which involves the confinement of a patient within, or movement of a patient through, a patient volume, particular reference will be made to MRI imaging systems (‘scanners’) by way of example only. It should be understood, however, that the present invention extends to all such medical systems.

For cost and other practical reasons, the radii of patient volumes such as 30 tend to be sufficiently small to cause, or enhance, anxiety in some patients and there is little point in having compact and efficient scanners which are economical to operate if significant numbers of patients cannot be persuaded to be scanned by them, or can only be so persuaded after much time consuming placation.

In accordance with one example of this invention, a frontal surface 40 of the scanner 10 surrounding the patient volume 30 is fitted with a plurality of sector-shaped display screens such as illustrated at 51, 52, 53, 54 and 55.

The screens 51 to 55 are preferably flat screens utilising plasma or LCD technology and, in this example, high-resolution LCD screens are used.

In order to calm patients by giving them the impression that the patient volume 30 is larger than it actually is, the screens 51 to 55 are fed, in this example, with video signals depicting the environment 60 behind the scanner 10 and looking away from it, to create an illusion of transparency or invisibility in the region surrounding the patient volume 30. Thus if, say, the scanner is located in a room and there is a wall of a certain colour behind it, the wall colour is imaged and depicted on the screens. It is of assistance in some circumstances in establishing the illusion if the wall is of a characteristic colour and/or has a particular pattern thereon, since the recognition of such visual stimuli may subconsciously assure the patient that the displayed image displayed is really the wall.

In this example, a wall 61 behind the scanner 10 includes a window 62 with curtains 63, 64. As can be seen in the drawing, a part 65 of the window 62, together with a portion 66 of the curtain 63, is actually obscured by the scanner from the view of a person approaching the front of the scanner. As indicated in the drawing, an image of that obscured part 65 of the window, together with the obscured part 66 of the curtain 63, is shown at 65′, 66′ on a relevant portion of the screen array 51 to 55 so as to appear in its proper relationship to the remainder of the window 62 which can be seen directly.

Of course, depending upon the perspective of the patient with reference to the scanner 10, it may be possible that the viewed environment behind the scanner 10 could include elements other than the wall or features thereon; for example a carpet or ceiling area, and/or some other equipment or features and, in such circumstances, the images displayed by various of the screens are varied to show what would actually be viewable by the patient were the scanner 10 not there. Similarly, the patient would also be able to see anyone and anything passing behind the scanner 10.

The video signals fed to the screens may be derived directly from a camera (effectively “live”) or they may be pre-recorded onto any convenient medium, such as a digital versatile disc (DVD) and played back as and when necessary by means of a suitable disc player, or other reproducer device. In alternative embodiments, artificial images may be produced by any suitable device, for example computer generated images. Such artificial images my include representations of the environment of the scanner, similar to the images produced by the cameras suggested above. Alternatively, the artificial images may represent an imaginary environment of the scanner, or indeed any image considered comforting to the patient.

In any case, there may be advantage in applying the video signals from the camera, the artificial image generating device or the player to the screens 51 to 55 by way of a digital processing circuit (DSP) capable of performing geometric and/or temporal processing upon the video signals. The DSP may be used, for example, to take account of differences in perspective or scale (from the patient's viewpoint) as between the image viewed on the screens and the scene that would be directly viewable if the scanner 10 were not there. This will need information on the position of the patient's head, and their direction of view. The simplest way of achieving this would be to arrange for the patient to approach the scanner through a relatively narrow corridor, effectively constraining the patient's position and line of sight.

The screens 51 to 55 may be replaced, if preferred with a single, annular screen, or other screen arrangements may be used if preferred, subject to surrounding the patient volume 30 with sufficient screen area to create the desired calming illusion.

Neighbouring screens may abut directly so as to present, so far as is practical, a substantially continuous image. Alternatively, neighbouring screens may be separated by finite distances and optical devices such as mirrors or 3-D pattern generating devices may be utilised to blur or disguise consequent gaps in the overall image.

Referring now to FIG. 2, there is shown an arrangement which may be used instead of, or in conjunction with, the arrangement of FIG. 1.

FIG. 2 shows a schematic and partial cross-section through the scanner 10, taken centrally and along the axis of the patient volume 30. All features similar to those of FIG. 1 carry the same reference numbers, and it will be appreciated that only features relevant to the understanding of the invention are included. For example, no attempt is made to show magnets or cryogenic systems.

In FIG. 2, a curved television screen 90, for example an LCD screen, is disposed above the patient support 20 and extends along the axis of the patient volume 30, and curves around that axis to an extent determined by the field of view of a patient lying on the support. The screen 90 may, as described with reference to FIG. 1, be fed with live or pre-recorded video signals depicting the environment beyond the scanner 10; in this case, of course, the ceiling and walls of the scanner room and any characteristic objects associated with the ceiling or walls or depending therefrom may be displayed to provide the desired effect of transparency or invisibility of the equipment.

In the case where live images are displayed on the screen, the patient would also be able to anyone and anything entering the room, for example if a radiologist, doctor or other carer came in to adjust patient position etc the patient would see the carer as well as feel the intervention.

Alternatively, or in addition, the video signals fed to screen 90 may comprise images intended to calm the patient, or otherwise distract the attention of the patient from the immediate environment of the patient volume 30. Thus artificial images such as patterns or graphics chosen to be calming might be used, either alone or superimposed upon live, recorded or artificial images of the ceiling and walls of the scanner room. Other distracting materials may be used, however, such as artistic or sporting images, or cartoons; those used in any instance being selected, for example, in dependence upon the psychological profiles of individual patients.

Instead of a single screen 90, a plurality of abutting screens may be used if preferred; in which case multiple screens may be disposed along and/or around the axis of the patient volume 30. Moreover an along-axis junction between any two neighbouring screens 90 may or may not align with a junction between any two of the neighbouring front-facing screens 51 to 55 (see FIG. 1).

Some medical apparatus, such as MRI scanners, are very sensitive to RF signals. Depending on the type of screen used, it may be necessary to provide RF screening to prevent any RF signals generated by the display screens from interfering with the operation of the medical apparatus.

1. An arrangement for presenting visual images to a patient to be investigated by a medical apparatus having a patient volume intended to accommodate at least part of the patient,
the arrangement comprising:
one or more cameras oriented along a line of sight of a patient when at least partially accommodated within the patient volume, or when approaching the medical equipment;
visual display means including at least one screen so disposed in relation to said medical apparatus as to be visible to the patient when looking along the line of sight;
means for supplying images from the camera(s) to the screen(s), such the patient views the images from the camera when looking along the line of sight.

2. An arrangement according to claim 1 wherein the means for supplying images comprises a means for recording images from the camera(s) and for reproducing said recorded images.

3. An arrangement according to claim 1, wherein the patient volume has an axis, wherein said visual display means comprises one or more screens each extending both radially and circumferentially of said axis and deployed to at least partially surround an entrance to said patient volume.

4. An arrangement according to claim 3, wherein said at visual display means comprises a plurality of sector-shaped component screens.

5. An arrangement according to claim 1, wherein the means for supplying images further includes means for subjecting video signals from said at least one camera to geometric processing to allow for differences in parallax, scale and/or orientation between the real environment represented by the video signals and the image(s) shown on said visual display means.

6. An arrangement according to claim 1, further including means for subjecting said images to temporal processing.

7. An arrangement according to claim 1, wherein said visual display means comprises one or more screens installed within, and extending axially along, said patient volume.

8. An arrangement according to claim 7, wherein said at least one screen is configured to wrap around said patient volume to an extent determined by a field of view of said patient.

9. (canceled)
